(19) United States
(12) Patent Application Publication
(10) Pub. No.: US 2007/0167751 A1
(43) Pub. Date: Jul. 19, 2007

(54) Method and apparatus for vessel characterization

(76) Inventors: Ronald B Schilling, Los Altos Hills, CA (US); Steven Elliot Stupp, San Carlos, CA (US); Timothy A. Riener, Freemont, CA (US)

Correspondence Address: STEVEN ELLIOT STUPP;PARK VAUGHAN & FLEMING, LLP, 2820 FIFTH STREET, DAVIS, CA, 95618, US

(21) Appl. No.: 11/295,052

(22) Filed: Dec. 5, 2005

(51) Int. Cl. A61B 8/00

(52) U.S. Cl. 600/437

(57) ABSTRACT

Techniques for characterizing a narrowing of a vessel are described. A location of a narrowing in a vessel is determined. A first velocity profile on a first surface is determined. The velocity profile corresponds to a fluid moving in the vessel and the first surface is in a region where the fluid motion is substantially laminar. A set of velocity profiles corresponding to the fluid motion are determined. Respective velocity profiles in the set of velocity profiles are determined on respective surfaces downstream of the narrowing and approximately downstream of a region of turbulent flow. A first characteristic of the narrowing is determined in accordance with the first velocity profile and the set of velocity profiles.

### BACKGROUND

1. Field of the Invention

The present invention relates to vessel characterization techniques, and more specifically, to techniques for characterization of stenoses in blood vessels.

2. Related Art

Fluid flow is an integral component in a wide variety of systems, ranging from power plants to the human body. In the latter, blood flow through the vascular system provides a myriad of functions, including perfusion of organs and tissues, circulating nutrients and removing waste products. Interruption or disruption of blood flow, even temporarily, may result in tissue infarction or even sudden death.

A variety of phenomena may lead to a disruption of blood flow. These include problems with the heart (the pump) and/or problems in one or more blood vessels (the plumbing) in the vascular system. For example, an artery may be at least partially blocked by a thrombosis or an embolism. Such occlusions of one or more blood vessels are often associated with arteriosclerosis or peripheral vascular disease.

Arteriosclerosis is a chronic disease characterized by an abnormal thickening and hardening of the arterial walls over a period of time. As plaque or deposits build up on the arterial walls, the lumen or central opening in the artery progressively decreases. Disruption of blood flow, however, is often sudden, for example, due to the development and/or shedding of a blood clot.

Furthermore, the deposits that accumulate on the arterial walls are often inhomogeneous. As a consequence, there may be local regions or segments in arteries where the lumen is narrower. At such a location, known as a region of stenosis or stenosis (and henceforth referred to as a stenosis), the normal blood flow in a blood vessel is modified. The blood velocity increases upon entering the stenosis; upon exiting, there is a jet in a region of high-velocity flow (approximately centered on the stenosis) as well as eddies or vortices in an annual-shaped region of turbulent flow proximate to a radial boundary of the blood vessel. It is thought that stenoses may increase a likelihood of an event where blood flow is interrupted or disrupted. Since such an event can have drastic health consequences, detection and characterization of stenoses (for example, by determining a stenosis size or cross-sectional areas) is often recommended by physicians to guide diagnosis and therapy.

A variety of conventional techniques exist for monitoring the vascular system as a whole, and for detecting and characterizing stenoses in particular. These include invasive techniques such as arteriography, as well as non-invasive techniques such as magnetic resonance imaging (for example, angiography) and positron emission tomography. Even the non-invasive techniques, however, often utilize a contrast agent or radioactive solution that is injected into a patient. In addition, the conventional techniques may be costly. These issues may limit the use of these techniques.

Ultrasound imaging techniques, such as Doppler or pulsed-wave ultrasound, are non-invasive and can be cost effective. Unfortunately, the region of turbulent flow at the exit of the stenosis poses a problem. In conventional ultrasound systems, the minimum width of an ultrasound beam is often large enough to encompass both the turbulent and non-turbulent regions of flow proximate to the exit of a stenosis. This overlap may preclude detection of signals and, therefore, the quantitative analysis of the fluid flow and the proper characterization of the stenosis. While 2-dimensional or 3-dimensional transducer arrays may allow for a reduction of the width of the beam, such transducers arrays are often expensive.

There is a need, therefore, for improved, non-invasive and cost-effective techniques for characterizing vessels, such as stenoses in blood vessels.

### SUMMARY

Methods and systems for characterizing a narrowing in a vessel and overcoming the previously described challenges are described. In an embodiment of the method, a location of the narrowing is determined. A first velocity profile on a first surface is determined. The velocity profile corresponds to a fluid moving in the vessel and the first surface is in a region where the fluid motion is approximately laminar. A set of velocity profiles corresponding to the fluid motion are determined. Respective velocity profiles in the set of velocity profiles are determined on respective surfaces downstream of the narrowing and approximately downstream of a region of turbulent flow. A first characteristic of the narrowing is determined in accordance with the first velocity profile and the set of velocity profiles.

The respective surfaces may each be a respective pre-determined distance from the first surface. The first surface and/or the respective surfaces may be approximately perpendicular to an axis of the vessel.

The first characteristic may be an average or mean cross-sectional area of the narrowing or a shape of the narrowing.

The first velocity profile and the set of velocity profiles may be determined using a Doppler shift of at least a first carrier signal having at least a first carrier signal frequency. The first carrier signal frequency may be approximately within an inclusive band of frequencies between 1 and 30 MHz. The first carrier signal frequency may correspond to a frequency in an ultrasound band of frequencies.

In some embodiments, one or more widths of one or more beams used in determining the location, the first velocity profile and/or the set of velocity profiles are wider than a vena contracta associated with the narrowing.

In some embodiments, the set of velocity profiles includes at least a second velocity profile on a second surface and a third velocity profile on a third surface.

In some embodiments, a boundary of the region of turbulent flow is determined. The boundary may be determined using a backscattered signal. At least one of the set of velocity profiles may be windowed or filtered to exclude a contribution from the region of turbulent flow. The windowing may be performed in the spatial and/or frequency domains.

In some embodiments, the first velocity profile is subtracted from each velocity profile in the set of velocity profiles. In some embodiments, one or more moments corresponding to one of more of the velocity profiles in the set of velocity profiles are determined. In some embodiments, a turbulent kinetic energy metric downstream of the narrowing is determined.

In some embodiments, the first characteristic in a pre-determined set of data is selected in accordance with at least one of the set of velocity profiles. The first characteristic may be selected further in accordance with the first velocity profile. The first characteristic may be determined by interpolating between at least a first pre-determined velocity profile and a second pre-determined velocity profile.

In another embodiment, a device for characterizing the narrowing in the vessel is described. The system includes at least one processor, at least one memory and at least one program module. The program module stored in the memory and executed by the processor includes instructions corresponding to one or more embodiments of the method. The system may include a Doppler measurement apparatus. The Doppler measurement apparatus may include one or more ultrasound transducers, transmit electronics and receive electronics.

### BRIEF DESCRIPTION OF THE FIGURES

FIG. 1A is an illustration of an embodiment of a system for characterizing a vessel.

FIG. 1B is an illustration of an embodiment of a vessel.

FIG. 2 is a flow diagram illustrating an embodiment of a process for characterizing a vessel.

FIG. 3 is a flow diagram illustrating an embodiment of a process for characterizing a vessel.

FIG. 4 is an illustration of a time varying arterial pressure in a blood vessel.

FIG. 5A is an illustration of a simulated turbulent kinetic energy as a function of a stenosis size and a distance from a stenosis in an embodiment.

FIG. 5B is an illustration of the simulated turbulent kinetic energy as the function of stenosis size and the distance from the stenosis in an embodiment.

FIG. 6A is an illustration of a simulated kurtosis of velocity as a function of the stenosis size and distance from the stenosis in an embodiment.

FIG. 6B is an illustration of the simulated kurtosis of the velocity as a function of the stenosis size and the distance from the stenosis in an embodiment.

FIG. 7A is an illustration of a simulated velocity difference as a function of a radial distance across the vessel in an embodiment.

FIG. 7B is an illustration of the simulated velocity difference as a function of the radial distance across the vessel in an embodiment.

FIG. 8A is an illustration of a standard deviation in the simulated velocity difference as a function of the distance from the stenosis in an embodiment.

FIG. 8B is an illustration of the standard deviation in the simulated velocity difference as a function of the distance from the stenosis in an embodiment.

FIG. 9A is an illustration of a slope of the standard deviation in the simulated velocity difference as a function of the distance from the stenosis in an embodiment.

FIG. 9B is an illustration of the slope of the standard deviation in the simulated velocity difference as a function of the distance from the stenosis in an embodiment.

FIG. 10 is a block diagram of an embodiment of a device for characterizing the vessel.

FIG. 11 is a block diagram of an embodiment of a data structure.

Like reference numerals refer to corresponding parts throughout the drawings.

### DETAILED DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

Techniques and related systems for characterizing one or more vessels are described. The approach is well suited to determining one or more characteristics of at least one narrowing in at least one vessel. The characteristics may include a mean size or cross-sectional area of the narrowing as well as a shape or an axial symmetry of the narrowing. While the approach has numerous applications to characterizing a wide variety of vessels that are used to guide flowing fluids (such as plumbing, for example, in heating and/or cooling systems), blood vessels (such as at least one of the carotid arteries) are used as illustrative embodiment in the discussion that follows. In the blood vessels, at least the one narrowing is a stenosis. It should be understood, however, that the approach may be applied in the characterization of valvular stenosis, of septal defects with shunt flow and, more generally, to vessel obstruction associated with a wide variety of causes (including a thrombosis or an embolism).

In the techniques and related systems for characterizing a stenosis in a blood vessel, one or more signals or waves having one or more carrier frequencies in an ultrasound band of frequencies (henceforth, ultrasound waves) are used to determine a velocity profile on a first surface that is upstream or downstream from the stenosis. In some embodiments, this velocity profile, and others discussed below, may include the velocity at a plurality of pixels in a plane corresponding to a surface, such as the first surface. The blood flow at the first surface may be approximately laminar. Additional velocity profiles are determined on a plurality of surfaces downstream from the stenosis, with at least one velocity profile determined on each surface in the plurality of surfaces. The plurality of surfaces may be located downstream from a region of turbulent flow proximate to the stenosis. This extent of this region of turbulent flow may be determined based on the scattering of the one or more ultrasound waves.

At least a first characteristic, such as the mean size of the stenosis, may be determined in accordance with the first velocity profile and the additional velocity profiles. In some embodiments, at least the first characteristic may selected and/or determined using a predetermined set of data. In some embodiments, at least the first characteristic is determined by interpolating between at least a first pre-determined velocity profile and a second pre-determined velocity profile in the pre-determined set of data. In some embodiments, at least the first characteristic is determined by interpolating between at least a first pre-determined velocity characteristic and a second pre-determined velocity characteristic in the pre-determined set of data.

In an exemplary embodiment, the ultrasound waves are generated using a Doppler or pulsed-wave ultrasound system that includes one or more ultrasonic transducers (such as one or more piezoelectric transducers) for transmitting and/or receiving the one or more ultrasound waves. The first velocity profile and the additional velocity profiles may be determined using a Doppler shift of the one or more ultrasound waves having at least a first carrier signal frequency. In other embodiments, the one or more ultrasound waves may include a range of carrier frequencies. At least the first carrier frequency may be selected in accordance with one or more transmission characteristics of the blood vessel and/or surrounding tissue/structures. In an exemplary embodiment, the first carrier signal frequency may be approximately within an inclusive band of frequencies between 1 and 30 MHz. In another embodiment, the first carrier signal frequency may be approximately within an inclusive band of frequencies between 3-10 MHz. In another embodiment, the first carrier signal frequency may be approximately within an inclusive band of frequencies between 1-5 MHz.

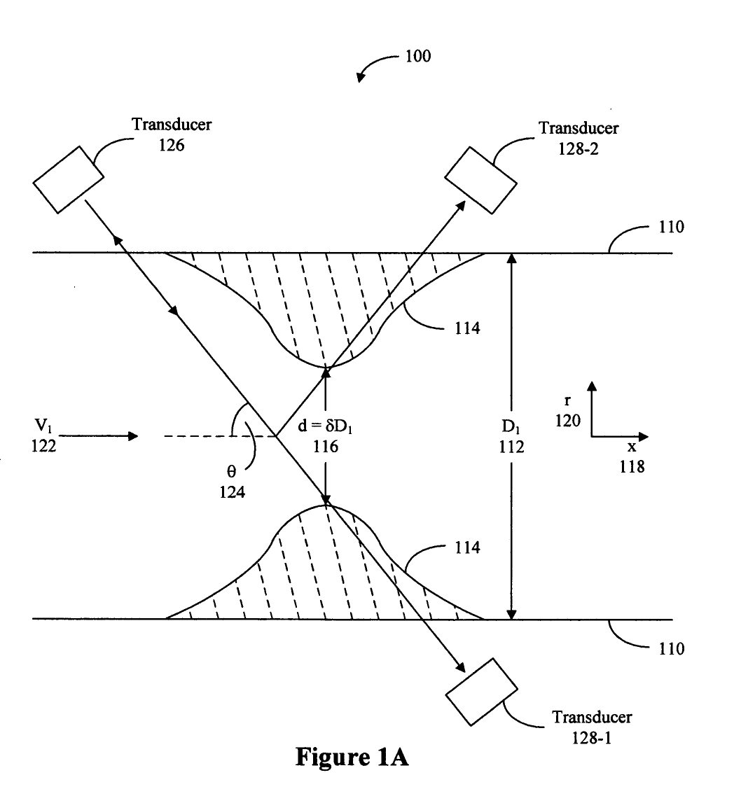

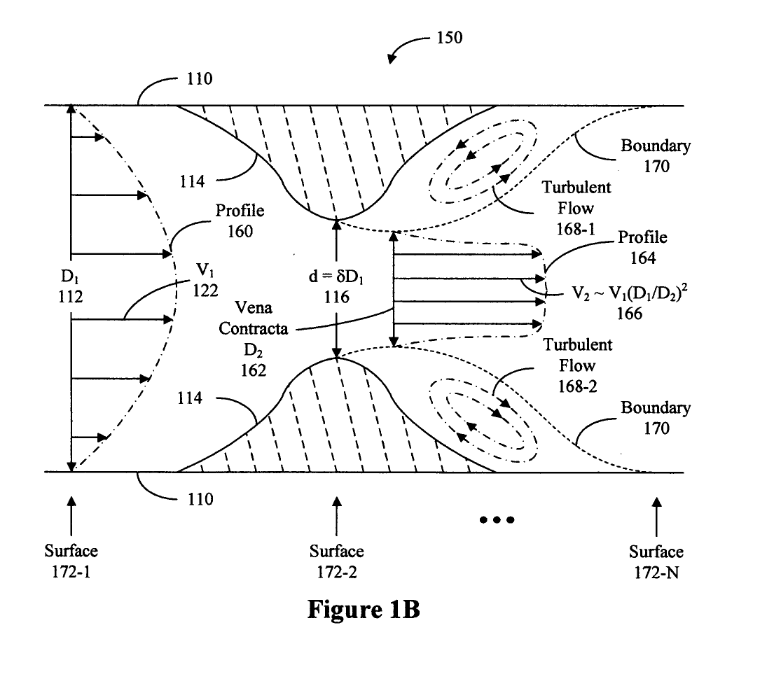

In some embodiments, the one or more beams may each have a minimum width that is larger than a vena contracta proximate to and associated with the stenosis. The vena contracta is described further below with reference to FIG. 1B. This capability may allow the techniques to be applied in systems and/or devices that utilize lower cost and readily available transducers.

Attention is now directed towards embodiments of techniques and related systems for characterizing vessels having a narrowing, such as a stenosis in a blood vessel. FIG. 1A is an illustration of an embodiment of a system 100 for characterizing a vessel 110 that includes a stenosis 114. The stenosis 114 at least partially occludes the vessel 110. The vessel 110 has a radial dimension 120 and an axial dimension 118.

In the system 100, a transducer 126 converts high frequency electrical signals into the one or more ultrasound waves. The transducer 126 transmits the one or more beams that include the one or more ultrasound waves. The one or more beams are transmitted at an angle θ 124 between an incidence direction of the one or more beams and an average direction of a blood velocity V₁ 122 in the vessel 110. In some embodiments, a reference co-ordinate system (for example, with respect to one or more anatomical landmarks) may be used to determine the angle θ 124. In some embodiments, one or more additional beams may be transmitted at additional angles. The one or more additional beams may be transmitted using the transducer 126 or one or more additional transducers (not shown).

The one or more beams ensonify a thin sample volume of blood flowing across a surface in the vessel 110. This surface, and others described below, may be approximately perpendicular to the axial dimension 118, i.e., approximately parallel to the radial dimension 120. The thin sample volume may be ensonified using a pulsed-wave or Doppler mode of operation in an ultrasound system, such as the system 100. In some embodiments, the sample volume may be in a region of flow that is approximately laminar. Blood within the sample volume reflects and/or scatters the one or more ultrasound waves. One or more reflected and/or scattered ultrasound waves are received by the transducer 126 (for backscattering measurements) and/or one or more transducers 128.

The one or more reflected and/or scattered ultrasound waves are converted into received electrical signals in the transducer 126 and/or the one or more transducers 128. The received electrical signals may be used to determine one or more characteristics of the stenosis 114, such as a mean cross-sectional diameter d 116. The mean diameter d 116 is a fraction δ of a mean diameter D₁ 112 of the vessel 110. The received electrical signals may include information corresponding to Doppler frequencies. Each Doppler frequency component in a spectrum of Doppler frequencies provides a measurement of an acoustic power that is proportional to a volume of scatterers in the sample volume that moved through the one or more beams at a corresponding velocity. For backscattering measurements, the Doppler frequency is given by

$$2 \frac{f}{c} V cos ( θ ) ,$$

where the factor of 2 is associated with round-trip propagation path differences, f is the carrier frequency of an ultrasound wave, c is a speed of sound (ranging from 1470 m/s in water to 4800 m/s in bone), V is the velocity of the scatterers and θ is the angle θ 124. Note that the scatterers in the sample volume are mainly red blood cells. The concentration of red blood cells is related to the blood volume by the hematocrit.

The scattering coefficient for the scatterers in the sample volume is a non-linear function of the hematocrit. In addition, the scattering coefficient is a function of the angle θ 124. Since the Doppler frequency is a function of the cosine of the angle θ 124 (as shown above), if a power spectrum of a demodulated received electronic signal is determined (for example, using an FFT algorithm) the corresponding velocity amplitude will vary as the cosine of the angle θ 124 and a width of the spectrum of Doppler frequencies will vary as an inverse of the cosine of the angle θ 124.

A thickness of the sample volume may be defined using range gating of the one or more reflected and/or scattered ultrasound waves (or the corresponding received electrical signals after transduction) that are received at the transducer 126 and/or the one or more transducers 128. A lateral dimension of the sample volume may correspond to widths of the one or more beams. These, in turn, may be an inverse function of an aperture of the one or more transducers, such as the transducer 126.

In some embodiments, one or more transducers in the system 100, such as the transducer 126, may include a 1-dimensional, a 1.5-dimensional, a 2-dimensional and/or a 3-dimensional array of transducer elements. (A 1.5 dimensional array may include 5 array elements in an elevation dimension of the array and a large number of elements, for example, 64, in a lateral dimension of the array.) A shape of the one or more beams may be modified using a mechanical lens, defocusing, electronic steering, electronic focusing and/or apodization (for phased-array transducers). In some embodiments, the system 100 uses electronic beam forming when receiving the one or more reflected and/or scattered ultrasound waves at one or more transducers, such as the transducer 126, to implement electronic steering and/or focusing.

In an exemplary embodiment, the one or more ultrasound waves transmitted by the one or more transducers, such as the transducer 126, are gated sine wave pulses that include between 6-12 periods of the sine waves. A pulse repetition rate of the one or more ultrasound waves may be chosen so as to avoid velocity and spatial aliasing, as is known in the art. Spatial aliasing may also be reduced by focusing the one or more ultrasound waves that are transmitted and/or the one or more reflected or scattered ultrasound waves that are received at a depth of focus corresponding to the sample volume (using techniques such as those described in the previous paragraph). This provides spatial discrimination with respect to regions in the vessel 110 that are not in focus.

In addition, if a received electrical signal does not correspond to blood flow, it may be excluded from the spectrum of Doppler frequencies using velocity filtering in the system 100. Such filtering may also be used to determine the spectrum of Doppler frequencies and/or to exclude Doppler frequencies that are associated with turbulent flow in the vessel 110. For example, velocity filtering may exclude contributions from residual turbulence, as well as scattering associated with tissue, which may have a scattered ultrasound power that is 100-1000 times greater than the ultrasound power associated with scattering by the blood. The latter may be accomplished using high-pass filtering. Filtering and/or averaging may be used to smooth the received electrical signals (to remove or reduce noise). For example, measurements may be performed a plurality of times (with each measurement corresponding to a time interval) and then averaged to improve a signal-to-noise ratio.

While the system 100 has been described in terms of pulse-wave of Doppler ultrasound, it may also support a continuous wave (CW) mode of operation, where one or more ultrasound waves are transmitted continuously. In this case, a pressure field at a receive transducer, such as the transducer 128-1, at any time may be attributed to scattering throughout the path of propagation of the one or more ultrasound waves. The system 100 may also support ultrasound scanning or sonography (also referred to as real-time imaging).

The system 100 may include fewer or additional components. Two or more components may be combined and/or a position of two or more components may be reversed. At least a portion of a function associated with one or more components may be performed by one or more other components.

FIG. 1B is an illustration an embodiment 150 of the vessel 110. The blood flow on a surface 172-1 upstream of the stenosis 114 is approximately laminar. In some embodiments, the blood flow at the surface 172-1 has a velocity profile 160 that is approximately parabolic. In larger vessels, however, the flow may have higher velocities, and thus a higher mean velocity V₁ 122. As a consequence, in such embodiments the velocity profile 160 may be blunter (i.e., non-parabolic). The flow at the surface 172-1 in such larger vessels, however, may still be approximately laminar. More generally, therefore, a region of laminar flow may be characterized by a ratio of a velocity bandwidth (corresponding to the profile 160) to a power-weighted mean velocity (corresponding to the mean velocity V₁ 122).

As the blood flows through the reduced mean cross-sectional area of the stenosis 114 it accelerates. A higher velocity stream or jet emerges downstream from the stenosis 114. For an axially symmetric stenosis 114 that is centered on the axial dimension 118 (FIG. 1A) of the vessel 110, the jet is centered on the axial dimension 118 (FIG. 1A). Dissipation at larger values in the radial dimension 120 (FIG. 1A) gives rise to an annular-shaped region of turbulence flow 168 further downstream from the stenosis 114. The turbulence flow 168 often includes eddies or vortices. There is typically a boundary 170 between the region of turbulent flow 168 and the jet. The turbulence flow 168 eventually dissipates, and for a surface sufficiently downstream from the stenosis 114 the flow once again has the mean velocity V₁ 122 and the velocity profile 160.

The jet has several distinguishing characteristics, including a vena contracta D₂ 162. The vena contracta D₂ 162 is a diameter corresponding to a smallest mean cross-sectional area traversed by the flow downstream from the stenosis 114. The vena contracta D₂ 162, therefore, corresponds to the highest flow velocities. Relative to the velocity profile 160, a velocity profile 164 at the vena contracta D₂ 162 is blunter toward the center with a steeper change in velocity at the boundary 170. An increase in velocity in the jet is proportional to an inverse of the stenosis size. A mean velocity of the jet V₂ 166 at the vena contracta D₂ 162 is approximately equal to V₁(D₁/D₂)². The flow is approximately laminar in the jet. Stated differently, for a given axial location or surface proximate to the stenosis 114, the jet is a region along the radial dimension 120 (FIG. 1A) where turbulence is at a minimum.

The system 100 (FIG. 1A) may be used to determine velocity profiles on a plurality of surfaces 172. In some embodiments, the plurality of surfaces 172 may have approximately an equal spacing from the stenosis 114, the surface 172-1 and/or another surface, such as surface 172-N. At least one of these velocity profiles, such as that on the surface 172-1, may be used to determine a reference or background velocity profile corresponding to a region of approximate laminar flow. In other embodiments, the background velocity profile may not correspond to a region of approximate laminar flow. As discussed further below, at least a subset of the other velocity profiles may be analyzed relative to one or more such background velocity profiles and used to determine one or more characteristics of the stenosis 114. For example, the background velocity profile may be subtracted from each velocity profile in at least the subset of the other velocity profiles.

The system 100 (FIG. 1A) may be used to determine an approximate location of the stenosis 114 (as illustrated by a surface 172-2). Since turbulence increases the backscattering of the one or more ultrasound waves, the system 100 (FIG. 1A) may also be used to determine the boundary 170 and/or the region of turbulent flow 168.

As discussed previously, the nature of the flow proximate to and downstream from the stenosis 114 may complicate or prevent the accurate quantitative determination of the one or more characteristics of the stenosis 114. In particular, there is the turbulent flow 168. In addition, the jet may entrain blood, i.e., the blood may become caught up in the jet thereby disturbing the blood flow. As a consequence of such effects, in some embodiments at least the subset of the velocity profiles that are used to determine the one or more characteristics of the stenosis 114 do not include contributions from the turbulent flow 168. As discussed previously, this may be accomplished using velocity filtering or windowing of the received electrical signals. In some embodiments, the filtering or windowing may be performed in a spatial domain. In some embodiments, the velocity profile measurements are performed on surfaces downstream from the region of turbulent flow 168. This may allow widths of one or more beams to be larger than the vena contracta D₂ 162.

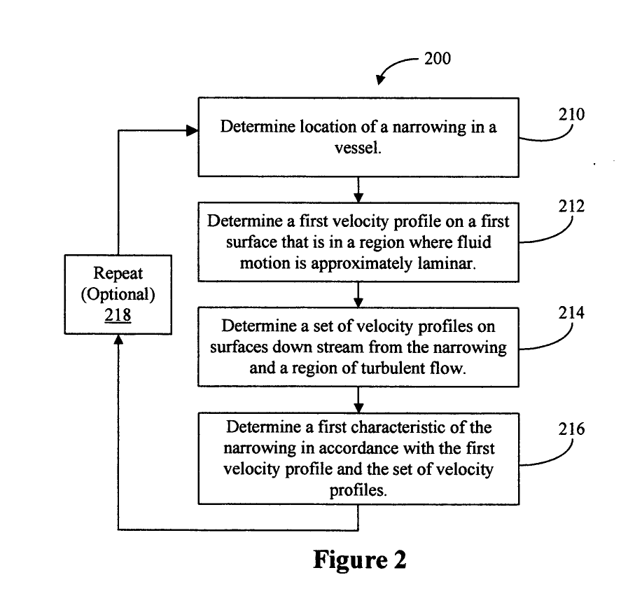

FIG. 2 is a flow diagram illustrating an embodiment of a process 200 for characterizing a vessel, such as the vessel 110 (FIGS. 1A and 1B). A location of a narrowing in a vessel is determined (210). A first velocity profile is determined in a region where fluid motion is approximately laminar (212). A set of velocity profiles are determined on surfaces downstream from the narrowing and a region of turbulent flow (214). A first characteristic of the narrowing is determined in accordance with the first velocity profile and the set of velocity profiles (216). These operations may be optionally repeated (218). The process 200 may include fewer or additional operations, two or more operations may be combined, and/or an order of two or more operations may be changed.

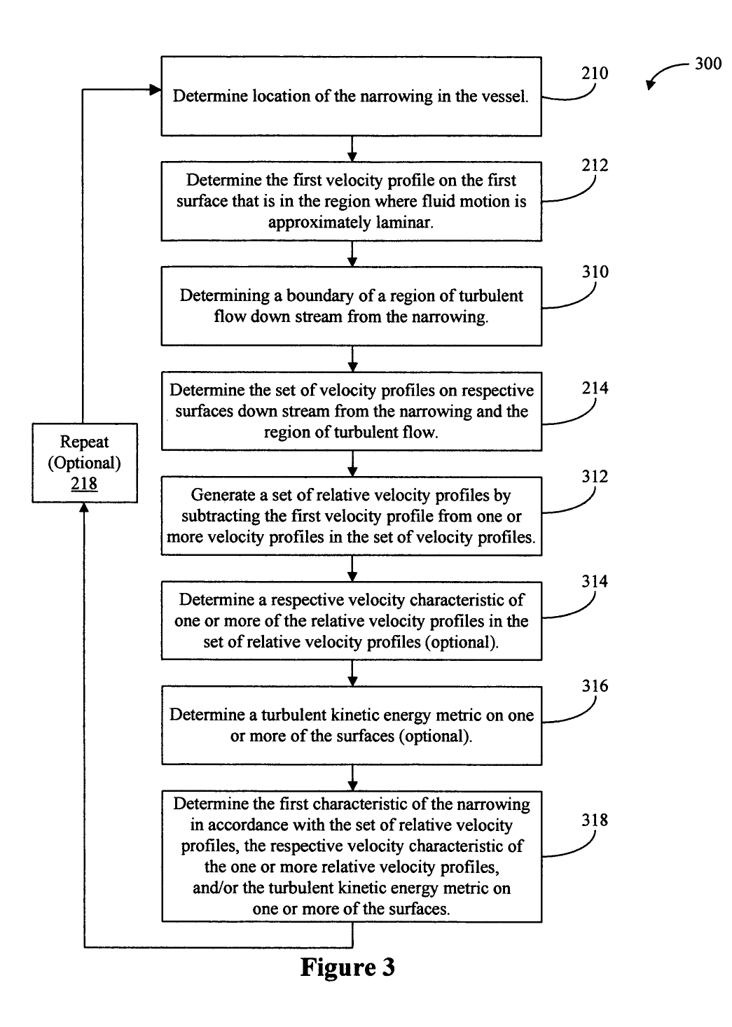

FIG. 3 is a flow diagram illustrating an embodiment of a process 300 for characterizing a vessel, such as the vessel 110 (FIGS. 1A and 1B). The location of the narrowing in the vessel is determined (210). The first velocity profile is determined in the region where fluid motion is approximately laminar (212). A boundary of a region of turbulent flow downstream from the narrowing is determined (310). The set of velocity profiles are determined on surfaces downstream from the narrowing and the region of turbulent flow (214). A set of relative velocity profiles are generated by subtracting the first velocity profile from one or more of the velocity profiles in the set of velocity profiles (312). A respective velocity characteristic (such as one or more moments of a distribution of velocities or a standard deviation in the distribution of velocities) of one of the relative velocity profiles is optionally determined (314). A turbulent kinetic energy metric (such as a root mean square of the noise at the boundary 170 in FIG. 1B or the root mean square of an amplitude of the power spectrum of the noise) on one or more of the surfaces is optionally determined (316). A first characteristic of the narrowing is determined in accordance with the set of relative velocity profiles, the respective velocity characteristic of the one or more relative velocity profiles, and/or the turbulent kinetic energy metric on one or more of the surfaces (318). These operations may be optionally repeated (218). The process 300 may include fewer or additional operations, two or more operations may be combined, and/or an order of two or more operations may be changed.

In an alternate embodiment, a set of relative velocity profiles (generated by subtracting the background velocity profile from the set of velocity profiles) may be used in overdetermined integral equation (corresponding to the Naiver-Stokes equation) to determine an approximate size and/or shape of a source region of the flow downstream from the stenosis 114 (FIGS. 1A and 1B), which in this case corresponds to the stenosis 114 (FIGS. 1A and 1B). In another embodiment, the set of velocity profiles may be used in the integral equation without subtracting the background velocity profile.

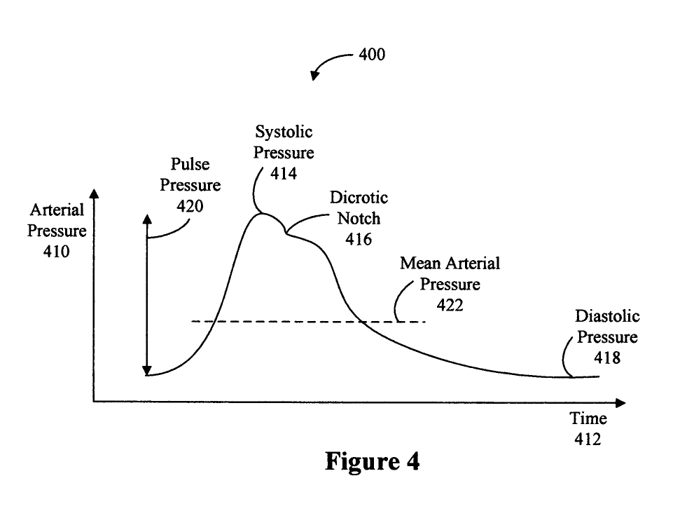

The discussion so far has implicitly treated the walls of the vessel 110 (FIGS. 1A and 1B) as being rigid. For blood vessels, this may be incorrect. The beating of the heart produces a pressure wave that propagates through the vascular system. This is illustrated in FIG. 4, which shows an arterial pressure 410 as a function of time 412. The time-varying pressure includes a systolic pressure 414, a dicrotic notch 416 and a diastolic pressure 418. A difference between the systolic pressure 414 and the diastolic pressure 418 is a pulse pressure 420. A mean arterial pressure 422 (approximately equal to the diastolic pressure 418 plus one-third of the systolic pressure 414) gives rise to a net velocity of the blood. Due to a finite rigidity, the walls of the blood vessels move in response to the pressure wave. This motion of the walls adds noise to Doppler ultrasound measurements.

To reduce or eliminate this noise source, the Doppler ultrasound measurements, for example, using the system 100 (FIG. 1A), may be gated based on one or more pre-defined points on an electrocardiogram (ECG) cycle of a patient, which measures electrical activity of the heart, and therefore corresponds to the pressure wave that propagates through the vascular system.

Attention is now directed towards an illustrative simulation of several metrics, such as the respective velocity characteristic 314 and the turbulent kinetic energy metric 316 in FIG. 3, as a function of the velocity V₁ 122 (FIGS. 1A and 1B) and the mean size of the stenosis 114 (FIGS. 1A and 1B). As discussed previously in the process 300 (FIG. 3), one or more of these metrics may be used to determine one or more characteristics of the stenosis 114 (FIGS. 1A and 1B). For example, one or more of the metrics may be determined for velocity profiles on a plurality of surfaces downstream from the stenosis 114 (FIGS. 1A and 1B) and the one or more stenosis characteristics may be looked up or determined (for example, by interpolation) in a table or data structure of predetermined velocity profiles and/or pre-determined metrics in accordance with the one or more metrics.

Computational fluid dynamics simulations were performed using commercially available computational fluid dynamics software. In these simulations, the stenosis 114 (FIGS. 1A and 1B) was axially symmetric and co-centric with the axial dimension 118 (FIG. 1A) of the vessel 110 (FIGS. 1A and 1B). It should be understood, however, that the results may be generalized to the non-axially symmetric and/or non-co-centric cases. The vessel 110 (FIGS. 1A and 1B) was cylindrical in shape and the vessel walls were rigid. This approximates the case where a respective point on the patient's ECG is used to gate the Doppler ultrasound measurements, as discussed above. The fluid pressure was 16 mN/mm² (corresponding to 120 mm-Hg). The mean velocity V₁ 122 (FIGS. 1A and 1B) was between 100-800 mm/s. The mean diameter D₁ 112 (FIGS. 1A and 1B) of the vessel 110 (FIGS. 1A and 1B) was 5 mm. The mean cross-sectional diameter d 116 (FIGS. 1A and 1B) of the stenosis 114 (FIGS. 1A and 1B) was between 1.6-3.6 mm (28-68% occlusion). The fluid density was 1008 kg/m³. The fluid viscosity was 2.7 cP. The boundary condition at the vessel walls was zero velocity.

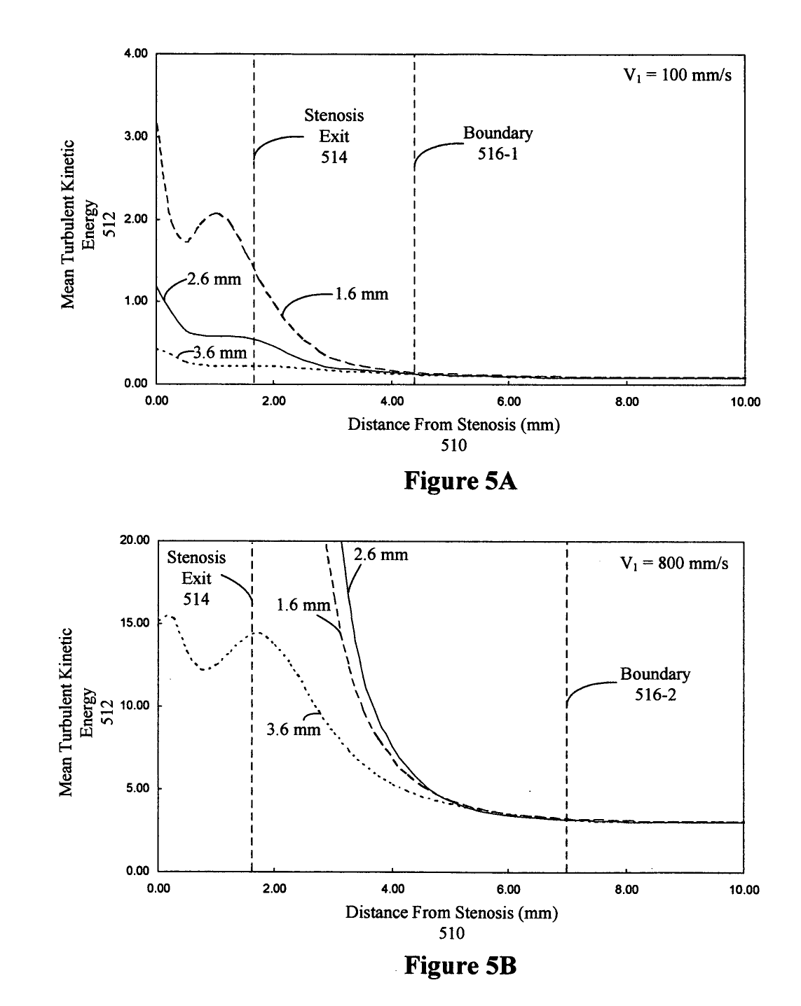

FIG. 5A is an illustration of a simulated turbulent kinetic energy 512 as a function of a stenosis size (1.6-3.6 mm) and a distance from the stenosis 510 in an embodiment with a mean velocity V₁ 122 (FIGS. 1A and 1B) of 100 mm/s. The turbulent kinetic energy is an estimate of the mean energy associated with turbulence. As noted in the discussion of the process 300 (FIG. 3), a turbulent kinetic energy or turbulent kinetic energy metric may include a root mean square of the noise at the boundary 170 in FIG. 1B and/or the root mean square of an amplitude of the power spectrum of the noise.

In general, as expected the turbulence increases for smaller mean stenosis sizes. And the turbulence decreases between a surface 514 corresponding to an exit from the stenosis and a boundary 516-1, which corresponds to the boundary 170 in FIG. 1B.

FIG. 5B is an illustration of the simulated turbulent kinetic energy 512 as a function of the stenosis size and the distance from a stenosis 510 in an embodiment with a mean velocity V₁ 122 (FIGS. 1A and 1B) of 800 mm/s. Note that boundary 516-2 is further downstream than the boundary 516-1 (FIG. 5A).

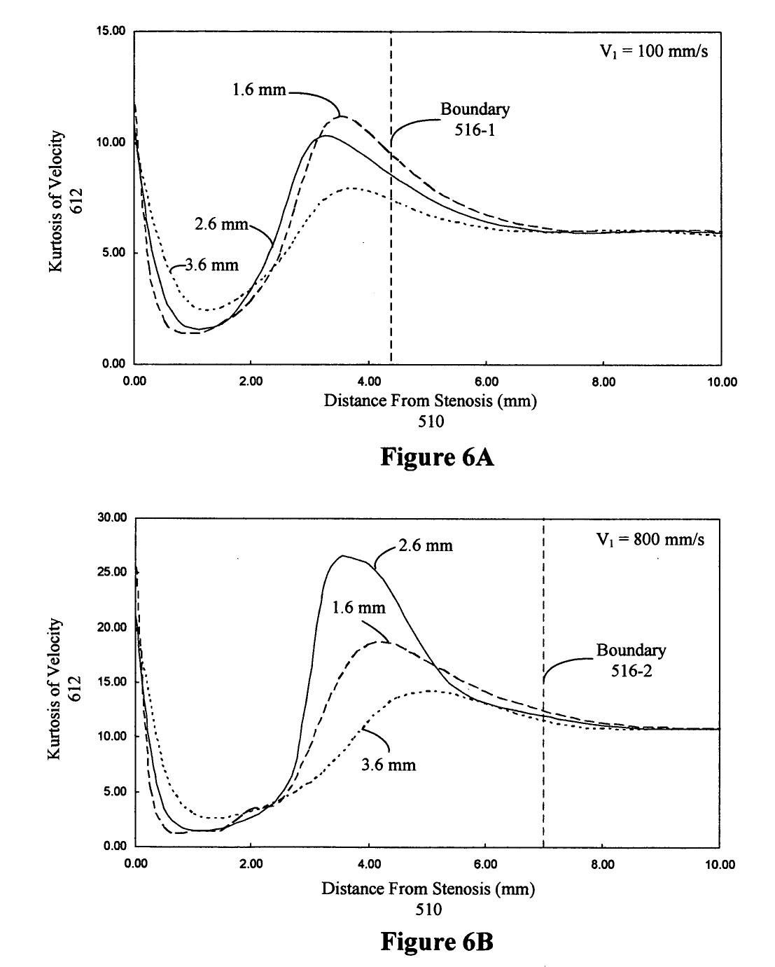

While the turbulent kinetic energy 512 in FIGS. 5A and 5B varies as a function of the distance 510 and the stenosis size, and therefore (at least in principle) could be determined on the plurality of surfaces downstream from the stenosis 114 (FIGS. 1A and 1B) and then used to determine a characteristic of the stenosis 114 (FIGS. 1A and 1B) such as the stenosis size, in practice this may be difficult. Therefore, in an exemplary embodiment a metric of the turbulence, such as the turbulent kinetic energy 512 or the root mean square noise, is used to determine the boundaries 516. Velocity profiles and/or velocity characteristics of the velocity profiles may be determined on surfaces downstream from a respective boundary, such as the boundary 516-2. The ability to use this information, as well as the location of the respective boundary, to determine the stenosis size is illustrated in FIGS. 6-9.

FIGS. 6A and 6B are illustrations of a simulated kurtosis 612 of velocity as a function of the stenosis size and the distance from a stenosis 510 in embodiments with mean velocities V₁ 122 (FIGS. 1A and 1B) of 100 and 800 mm/s, respectively. For a respective surface corresponding to a respective distance from the stenosis 114 (FIGS. 1A and 1B), kurtosis is a velocity characteristic of the distribution of velocities corresponding to the velocity profile. In other embodiments, one or more additional velocity characteristics of the distribution of velocities on a respective surface (such as one or more moments of the distribution of velocities) may be used. In FIGS. 6A and 6B, there are differences in the kurtosis 612 as a function of the distance 510 downstream from the boundaries 516. Thus, velocity profile measurements and/or determination of a velocity characteristic, such as the kurtosis, may be used to determine the stenosis size. Alternatively, such measurements and analysis may be performed between the stenosis exit 514 (FIGS. 5A and 5B) and at least one of the boundaries 516. However, a narrower beam focus (to avoid regions with turbulent flow) and/or additional spatial and/or velocity filtering may be needed in such embodiments.

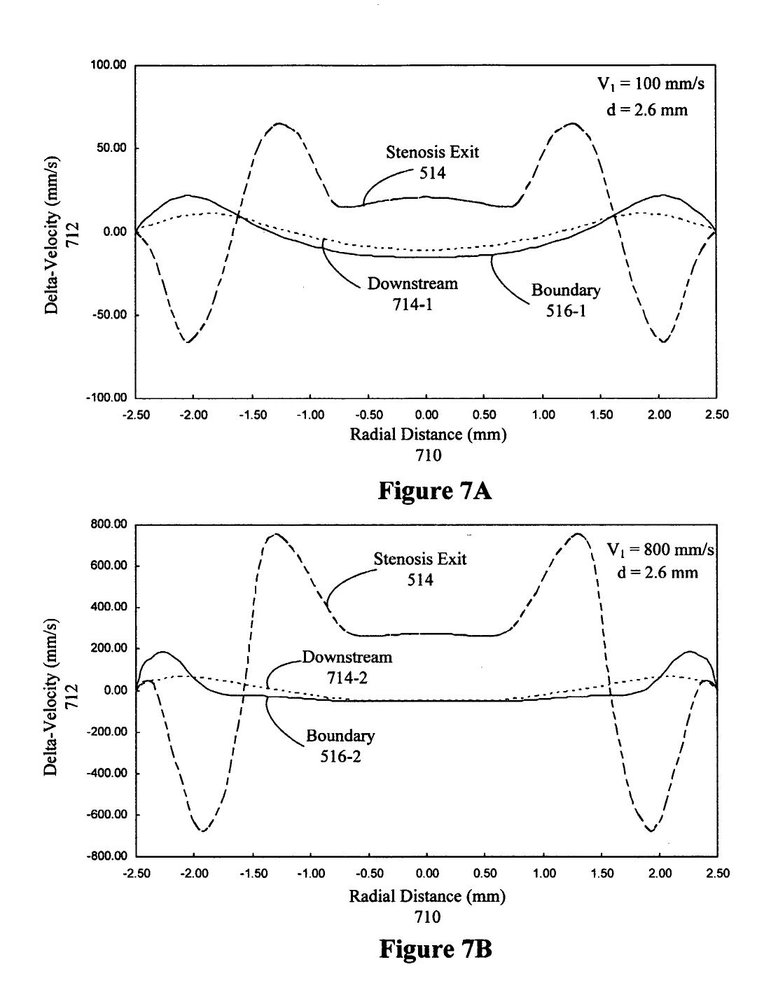

FIGS. 7A and 7B are illustrations of a simulated velocity difference or delta-velocity 712 (between a surface downstream from the stenosis 114 in FIGS. 1A and 1B and a surface downstream of the region of turbulent flow) as a function of a radial distance 710 across the vessel in embodiments with mean velocities V₁ 122 (FIGS. 1A and 1B) of 100 and 800 mm/s, respectively. In FIGS. 7A and 7B, the mean cross-sectional diameter d 116 (FIGS. 1A and 1B) of the stenosis 114 (FIGS. 1A and 1B) is 2.6 mm. Delta velocity curves are shown on a surface at the stenosis exit 514, at the boundary 516 and another surface further downstream 714 from the boundary 516. Note that this other surface 714 is distal from the boundary 516 but is upstream from a region where the flow has fully returned to its state in the absence of the stenosis 114 (FIGS. 1A and 1B), i.e., flow corresponding to the surface 172-1 in FIG. 1B. The delta-velocity curves indicate that the velocity profile has not fully relaxed proximate but downstream from the boundary 516.

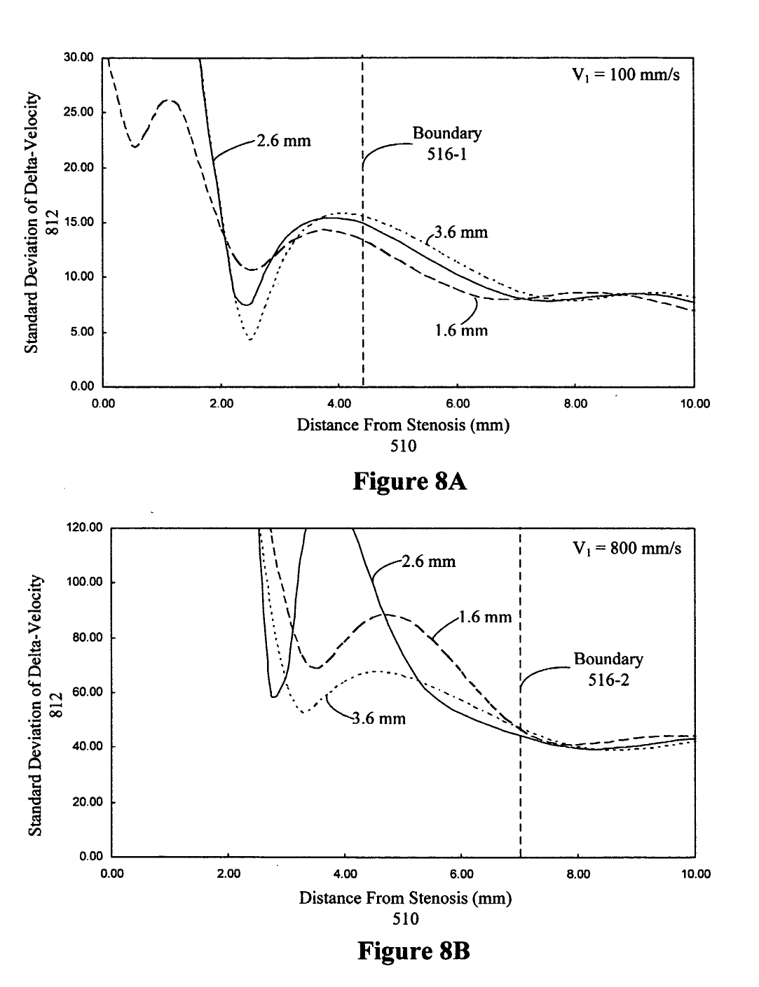

FIGS. 8A and 8B are illustrations of a standard deviation in a simulated velocity difference 812 as a function of the stenosis size and the distance from the stenosis 510 in embodiments with mean velocities V₁ 122 (FIGS. 1A and 1B) of 100 and 800 mm/s, respectively. Downstream of the boundary 516, the standard deviation in the simulated velocity difference 812 is dependent on the stenosis size. Velocity profile measurements performed on one or more surfaces in this region may be used to determine this velocity characteristic, which in turn, may be used to determine the stenosis size.

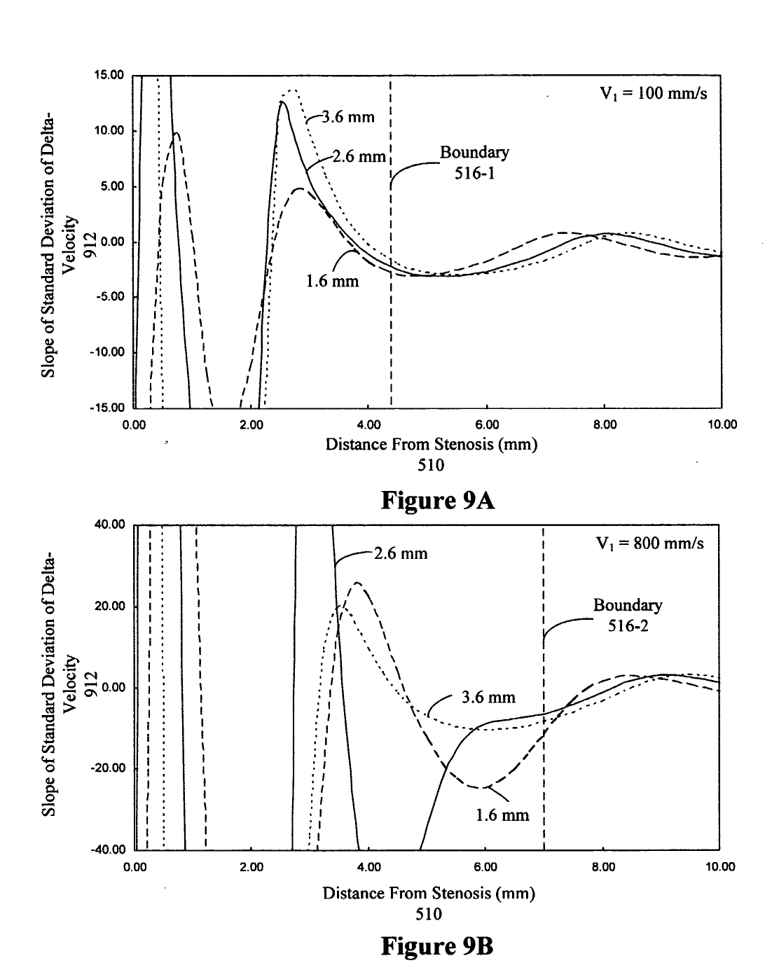

FIGS. 9A and 9B are illustrations of a slope or a first derivative of a standard deviation in a simulated velocity difference 912 as a function of the stenosis size and the distance from the stenosis 510 in embodiments with mean velocities V₁ 122 (FIGS. 1A and 1B) of 100 and 800 mm/s, respectively. Downstream of the boundary 516, the slope 912 is dependent on the stenosis size. Over a range of mean velocities V₁ 122 (FIGS. 1A and 1B) there is a maximum sensitivity downstream from the boundary 516 for a respective stenosis size. This velocity characteristic also appears to be less dependent on the mean velocity V₁ 122 (FIGS. 1A and 1B). Velocity profile measurements performed on one or more surfaces in this region may be used to determine this velocity characteristic, which in turn, may be used to determine the stenosis size.

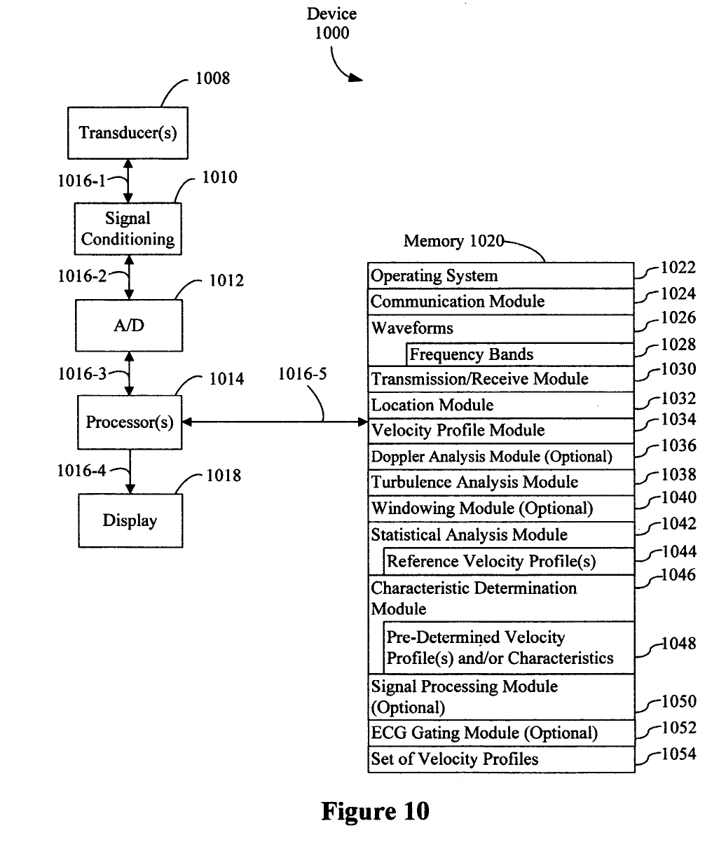

Attention is now directed towards embodiments of an ultrasound measurement device. FIG. 10 is a block diagram of an embodiment of a device 1000 for characterizing a vessel. The device 1000 includes one or more transducers 1008, a signal conditioning module 1010, an analog-to-digital (A/D) converter 1012, one or more processing units (CPUs) 1014, a display 1018, a memory 1020 (including primary and/or secondary storage), and one or more signal lines 1016 for connecting these components. In some embodiments, the one or more signal lines may include one or more communication buses. In alternate embodiments, some or all of the functionality of the device 1000 may be implemented in one or more application specific integrated circuits (ASICs), thereby either eliminating the need for one or more of one or more processing units 1014 or reducing the role of the one or more processing units 1014. The one or more processing units 1014 may support parallel processing and/or multi-threaded environments.

The one or more transducers 1008 may convert high frequency electrical signals into the one or more ultrasound waves in the one or more beams, which are transmitted. The one or more transducers 1008 may receive the one or more reflected and/or scattered ultrasound waves and convert these into received electrical signals.

The signal condition module 1010 may convert electrical signals from baseband to or from one or more high frequency electrical signals having one or more carrier frequencies. The signal condition module 1010 may perform amplification and filtering, and may optionally convert the electrical signals at baseband into the Doppler frequency spectrum.

The A/D 1012 may convert signals between analog and digital domains.

The display 1018 may be used to present information, such as the Doppler frequency spectrum and/or an ultrasound image, to a user of the device 1000.

The memory 1020 may include high speed random access memory (DRAM, SRAM) and may also include non-volatile memory, such as one or more magnetic disk storage devices, optical disk storage devices, flash memory devices and/or other non-volatile solid state storage devices. The memory 1020 may include mass storage that is remotely located from the one or more processing units 1014.

The memory 1020 may store an operating system (or a set of instructions) 1022 that includes procedures for handling various basic system services for performing hardware dependent tasks. The operating system 1022 may be an embedded operating system. The memory may also store a communications module (or a set of instructions) 1024 that is used for controlling the communication between the device 1000 and other devices, computers or servers. Communication may occur using one or more protocols, such as TCP/IP, a wireless protocol and/or an interface protocol (such as USB). Communication may occur on one or more networks, such as the Internet, a local area network, and/or a wireless networks (for example, a cellular telephone network, a Wi-Fi network and/or a Bluetooth network). The one or more networks may include those using infrared communication, optical communication and/or wireless communication.

The memory 1020 may store instructions or parameters for one or more waveforms 1026, in one or more frequency bands 1028, that may be generated by the device 1000. The memory 1020 may store a transmission/receive module (or a set of instructions) 1030. The transmission/receive module 1030 may include instructions and procedures for gating, steering and focusing of the one or more beams.

The memory 1020 may also store a location module (or a set of instructions) 1032 for determining the locations of one or more stenoses, a velocity profile module (or a set of instructions) 1034 for determining one or more velocity profiles on one or more surfaces, an optional Doppler analysis module (or a set of instructions) 1036 for determining the Doppler frequency spectrum from received electrical signals, a turbulence analysis module (or a set of instructions) 1038 for determining a boundary of the region of turbulent flow, an optional windowing module (or a set of instructions) 1040 for spatial and/or frequency filtering (for example, to exclude a contribution from the region of turbulent flow), a statistical analysis module (or a set of instructions) 1042 for at least partially analyzing one or more velocity profiles that are determined on one or more surfaces (which may include determining one or more velocity characteristics of a respective velocity profile), a characteristic determination module (or a set of instructions) 1046 for determining, interpolating, selecting or retrieving one or more characteristics of one or more stenoses, an optional signal processing module (or a set of instructions) 1050 for additional conditioning of electrical signals, an optional ECG gating module (or a set of instructions) 1052, and/or a set of velocity profiles 1054 that are measured on one or more surfaces using the device 1000.

The statistical analysis module 1042 may include one or more reference velocity profiles 1044 that may be subtracted from one or more of the velocity profiles 1054. The characteristic determination module 1046 may include one or more pre-determined velocity profiles and/or velocity characteristics 1048. The one or more characteristics of the one or more stenoses may be looked up in the one or more pre-determined velocity profiles and/or velocity characteristics 1048. In some embodiments, the one or more characteristics of the one or more stenoses may be determined by interpolating between the one or more pre-determined velocity profiles and/or velocity characteristics 1048.

The device 1000 may include fewer or additional components, modules and/or data structures. Two or more modules or components may be combined and/or an order of two or more modules or components may be changed. At least a portion of the function of at least one module or component may be implemented using one or more other modules or components. Functions that are implemented in hardware may be implemented, at least in part, in software. Functions that are implemented in software may be implemented, at least in part, in hardware.

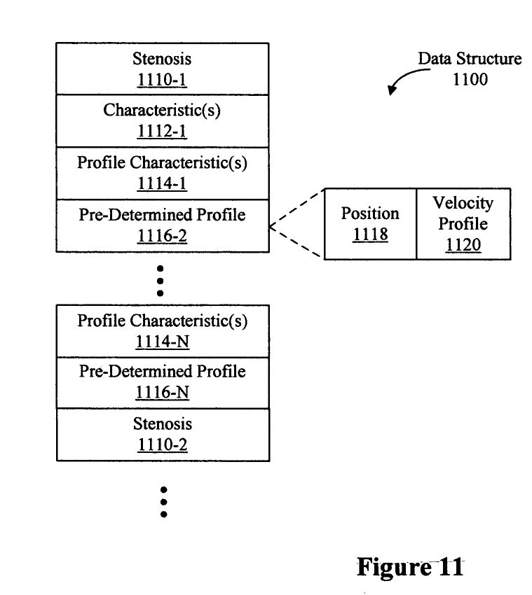

Attention is now directed towards embodiments of data structures that may be used in the device 1000 (FIG. 10) and/or the system 100 (FIG. 1A). FIG. 11 is a block diagram of an embodiment of a data structure 1100. The data structure 1100 may include a plurality of entries for a plurality of stenoses 1110. The entries for each stenosis, such as stenosis 1110-1, may include one or more characteristics 1112-1 of the stenosis 1110-1, optional pre-determined velocity characteristic(s) 1114 and corresponding pre-determined velocity profiles 1116. Each pre-determined velocity profile, such as pre-determined velocity profile 1116-1, may include a position (relative to the stenosis 1110-1 or another reference location) 1118 of a surface where a pre-determined velocity profile 1120 was determined or measured, as well as the pre-determined velocity profile 1120. The data structure 1100 may include fewer or more entries. Two or more entries may be combined into a single entry and a relative position of two or more entries may be changed.

The foregoing descriptions of embodiments of the present invention have been presented for purposes of illustration and description only. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

What is claimed is:

1. A method of characterizing a narrowing in a vessel, comprising:
determining a location of the narrowing;
determining a first velocity profile on a first surface, wherein the velocity profile corresponds to a fluid moving in the vessel and the first surface being in a region where the fluid motion is substantially laminar;
determining a set of velocity profiles corresponding to the fluid motion, wherein respective velocity profiles in the set of velocity profiles are determined on respective surfaces downstream of the narrowing and are approximately downstream of a region of turbulent flow; and
determining a first characteristic of the narrowing in accordance with the first velocity profile and the set of velocity profiles.

2. The method of claim 1, wherein the respective surfaces are each a respective pre-determined distance from the first surface.

3. The method of claim 1, wherein the first surface is substantially perpendicular to an axis of the vessel.

4. The method of claim 1, wherein the respective surfaces are substantially perpendicular to an axis of the vessel.

5. The method of claim 1, wherein the set of velocity profiles includes at least a second velocity profile on a second surface and a third velocity profile on a third surface.

6. The method of claim 1, wherein the first velocity profile and the set of velocity profiles are determined using a Doppler shift of at least a first carrier signal having at least a first carrier signal frequency.

7. The method of claim 6, wherein the first carrier signal frequency is substantially within an inclusive band of frequencies between 1 and 30 MHz.

8. The method of claim 6, wherein the first carrier signal frequency corresponds to a frequency in an ultrasound band of frequencies.

9. The method of claim 1, wherein the first characteristic is an average cross-sectional area of the narrowing.

10. The method of claim 1, wherein the first characteristic is a shape of the narrowing.

11. The method of claim 1, further comprising determining a boundary of the region of turbulent flow.

12. The method of claim 1, further comprising subtracting the first velocity profile from each velocity profile in the set of velocity profiles.

13. The method of claim 1, further comprising determining one or more moments corresponding to one of more of the velocity profiles in the set of velocity profiles.

14. The method of claim 1, further comprising determining a turbulent kinetic energy metric downstream of the narrowing.

15. The method of claim 1, further comprising selecting the first characteristic in a pre-determined set of data in accordance with at least one of the set of velocity profiles.

16. The method of claim 15, wherein the selecting is further in accordance with the first velocity profile.

17. The method of claim 1, further comprising determining the first characteristic by interpolating between at least a first pre-determined velocity profile and a second pre-determined velocity profile.

18. The method of claim 1, further comprising determining a boundary of the region of turbulent flow using a backscattered signal.

19. The method of claim 18, further comprising windowing at least one of the set of velocity profiles to exclude a contribution from the region of turbulent flow.

20. The method of claim 1, wherein one or more widths of one or more beams used in determining the location, the first velocity profile and the set of velocity profiles are wider than a vena contracta associated with the narrowing.

21. A computer-program product for characterizing the narrowing in a vessel, the computer-program product for use in conjunction with a computer system, the computer-program product comprising a computer-readable storage medium and a computer-program mechanism embedded therein, the computer-program mechanism comprising:
instructions for determining a location of the narrowing;
instructions for determining a first velocity profile on a first surface, wherein the velocity profile corresponds to a fluid moving in the vessel and the first surface being in a region where the fluid motion is substantially laminar;
instructions for determining a set of velocity profiles corresponding to the fluid motion, wherein respective velocity profiles in the set of velocity profiles are determined on respective surfaces downstream of the narrowing and are approximately downstream of a region of turbulent flow; and
instructions for determining a first characteristic of the narrowing in accordance with the first velocity profile and the set of velocity profiles.

22. A device for characterizing the narrowing in a vessel, comprising:
at least one processor;
at least one memory; and
at least one program module, the program module stored in the memory and executed by the processor, the program module containing:
instructions for determining a location of the narrowing;
instructions for determining a first velocity profile on a first surface, wherein the velocity profile corresponds to a fluid moving in the vessel and the first surface being in a region where the fluid motion is substantially laminar;
instructions for determining a set of velocity profiles corresponding to the fluid motion, wherein respective velocity profiles in the set of velocity profiles are determined on respective surfaces downstream of the narrowing and are approximately downstream of a region of turbulent flow; and
instructions for determining a first characteristic of the narrowing in accordance with the first velocity profile and the set of velocity profiles.

23. A computer mechanism for characterizing the narrowing in a vessel, comprising:
processor means for performing computations;
memory means; and
program module mechanism, the program module mechanism stored in the memory means and executed by the processor means, the program module mechanism containing:
instructions for determining a location of the narrowing;
instructions for determining a first velocity profile on a first surface, wherein the velocity profile corresponds to a fluid moving in the vessel and the first surface being in a region where the fluid motion is substantially laminar;
instructions for determining a set of velocity profiles corresponding to the fluid motion, wherein respective velocity profiles in the set of velocity profiles are determined on respective surfaces downstream of the narrowing and are approximately downstream of a region of turbulent flow; and
instructions for determining a first characteristic of the narrowing in accordance with the first velocity profile and the set of velocity profiles.
